(19) United States
(12) Patent Application Publication
(10) Pub. No.: US 2026/0013738 A1
(43) Pub. Date: Jan. 15, 2026

(54) Systems and Methods for Monitoring of Blood Pressure

(71) Applicant: Beckton, Dickinson and Company, Franklin Lakes, NJ (US)

(72) Inventors: Boris Reuderink, Amersfoort (NL); Hans Jean Paul Kuijkens, Amsterdam (NL); Jeroen Van Goudoever, Amsterdam (NL)

(21) Appl. No.: 18/994,438

(22) PCT Filed: Jul. 10, 2023
(86) PCT No.: PCT/US2023/027273

§ 371 Date: Jan. 14, 2025

Related U.S. Application Data
(60) Provisional application No. 63/389,141, filed on Jul. 14, 2022.

(51) Int. Cl. A61B 5/021; A61B 5/00

(52) U.S. Cl. CPC A61B 5/02108; A61B 5/7267

(57) ABSTRACT

Systems and methods for reconstructing radial arterial pressure are provided. Noninvasive arterial pressure data derived from a finger or thumb can be transformed into a radial arterial pressure. Computational models can be trained and utilized to predict the parameters to transform finger or thumb arterial pressure into radial arterial pressure.

### TECHNOLOGICAL FIELD

The disclosure is generally directed to systems and methods for monitoring blood pressure, and more specifically for determining the non-invasive radial blood pressure waveform, measured with a digit sensor.

### BACKGROUND

Continuous noninvasive blood pressure monitors enable real-time measurement of blood pressure waves and derived hemodynamic parameters. Multiple techniques can be utilized including the volume clamp method.

The volume clamp method measures arterial blood pressure at an extremity (e.g., finger) utilizing an inflatable cuff, a light source (e.g., light emitting diode (LED)), and light sensor. The pressure in the cuff is adjusted to keep the diameter of the artery constant (the unloaded state), in which the diameter is determined via the light source and light sensor. The pressure within the inflatable cuff represents the arterial pressure of the finger artery. The finger arterial pressure waveforms can be converted into brachial arterial pressure waveforms and central arterial pressure (e.g., aortic pressure) waveforms can be computed from the brachial arterial pressure waveforms.

### SUMMARY

Systems and methods for monitoring blood pressure can comprise utilization of a non-invasive sensor on a finger or thumb to compute a radial arterial pressure. The non-invasive sensor can comprise a pressurized cuff and a photoplethysmogram to obtain hemodynamic data and physiological data from the finger or thumb. An arterial pressure wave form of the finger or thumb can be transformed into a radial arterial pressure utilizing transfer function parameters selected via a lookup table or a computational model. The look up table can be generated via a computational model. Computational models can learn associations between hemodynamic data and physiological data from the finger or thumb and radial intra-arterial pressure.

In some implementations, a real-time method is for selecting a set of transformation function parameters for transforming digit arterial pressure data into radial arterial pressure data. The method comprises receiving, using a computational processing system, hemodynamic data and physiological data. The hemodynamic data and physiological data comprise sensor readings of a digit. The hemodynamic data comprise digit arterial pressure waveform data and the physiological data are physiological information of the digit.

In some implementations, the method comprises mapping, using a computational processing system, the hemodynamic data and physiological data to a vector representative of the hemodynamic data and physiological data.

In some implementations, the method comprises selecting, using a computational processing system, a set of transformation function parameters based on the representative vector. The set of transformation parameters can be utilized to transform the digit arterial pressure waveform data into radial arterial pressure waveform data.

In some implementations, the computational processing system is housed within a hemodynamic monitoring system.

In some implementations, the hemodynamic monitoring system comprises a pressurized digit cuff. The method further comprises obtaining a digit arterial pressure data via a volume clamp method.

In some implementations, the hemodynamic monitoring system comprises a photoplethysmogram. The method further comprises obtaining the sensor readings via the photoplethysmogram.

In some implementations, the hemodynamic data and physiological data further comprise one or more of: cardiac output, stroke volume, stroke volume variation, vascular tone, contractility, afterload, systemic vascular resistance, systolic pressure of a digit, diastolic pressure of a digit, mean arterial pressure of a digit, left ventricular ejection time, reconstructed brachial arterial waveform data, pulse pressure, DC, AC,

$$\frac{HR}{fSys} , \frac{fDia}{fMAP} , \frac{fSys - fDia}{fMAP} , \frac{fMAP - fDia}{fSys - fDia} , \frac{dP}{dt} , \frac{AC}{DC} ,$$

or instrumentation data comprising one or more: cuff type, cuff size, or sensor position in relation to heart.

In some implementations, the method further comprises determining, using the computational processing system, that a particular data point of the hemodynamic data and physiological data is beyond a limit threshold. The mapping step further comprises utilizing a limit value instead of the particular data point.

In some implementations, the mapping step comprises performing one of the following techniques: principal component analysis (PCA), kernel-PCA, graph-based kernel PCA, the Nyström method, linear discriminant analysis (LDA), generalized discriminant analysis (GDA), T-distributed stochastic neighbor embedding (t-SNE), or uniform manifold approximation and projection (UMAP).

In some implementations, the selecting step further comprises: selecting, using the computational processing system, the set of transformation function parameters via a lookup table comprising learned associations between feature vectors and transformation function parameters.

In some implementations, the method further comprises training, using the computational processing system, a computational model to generate the lookup table. The training comprises learning associations between a cohort of patient data and radial intra-arterial pressure measurements captured via catheterization. The patient data comprises hemodynamic data obtained from a pressurized digit cuff and physiological data obtained from a photoplethysmogram.

In some implementations, the selecting step further comprises: selecting, using the computational processing system, the set of transformation function parameters using a regression-based model or a classification-based model.

In some implementations, the method further comprises training, using the computational processing system, the regression-based model or the classification-based model by learning associations between cohort of patient data and radial intra-arterial pressure measurements captured via catheterization. The patient data comprises hemodynamic data obtained from a pressurized digit cuff and physiological data obtained from a photoplethysmogram.

In some implementations, the method further comprises determining, using the computational processing system, that a particular parameter of the selected set of transformation function parameters is beyond a limit threshold. The method further comprises selecting, using the computational processing system, an alternative parameter value to replace the particular parameter that is beyond the limit threshold.

In some implementations, the alternative parameter value is one of: an upper limit value or a lower limit value.

In some implementations, the method further comprises determining, using the computational processing system, the representative vector is abnormal. The selecting step further comprises: selecting, using the computational processing system, one of: a previously selected set of transformation function parameters, a set of transformation function parameters having a set of prespecified values, or a combination of previously selected sets of transformation function parameters.

In some implementations, the method further comprises transforming, using the computational processing system, the digit arterial pressure waveform data into radial arterial pressure waveform data utilizing the selected set of transformation function parameters.

In some implementations, the transforming step is performed by a linear transformation and the selected transformation function parameters are a scale and an offset. The method further comprises centering, using the computational processing system, the digit arterial pressure waveform data by subtracting an arterial pressure of the digit arterial pressure waveform data. The method further comprises scaling, using the computational processing system, the digit arterial pressure waveform data with the selected scale. The method further comprises uncentering, using the computational processing system, the scaled digit arterial pressure waveform data by adding the arterial pressure of the digit arterial pressure waveform data. The method further comprises adding, using the computational processing system, the selected offset to the uncentered and scaled digit arterial pressure waveform data to yield the radial arterial pressure waveform data.

In some implementations, the arterial pressure is one of: a mean arterial pressure, a systolic arterial pressure, or a diastolic arterial pressure.

In some implementations, the method further comprises displaying, using the computational processing system, the radial arterial pressure waveform data on a display screen.

In some implementations, the method further comprises determining, using the computational processing system, proximal arterial pressure waveform data from the radial arterial pressure waveform data. The method further comprises displaying, using the computational processing system, the proximal arterial pressure waveform data on a display screen.

In some implementations, a method is for transforming digit arterial pressure waveform data into radial arterial pressure waveform data. The method comprises selecting, using a computational processing system, a scale and an offset. The method comprises centering, using the computational processing system, digit arterial pressure waveform data by subtracting the mean arterial pressure of the digit arterial pressure waveform data. The method comprises, using the computational processing system, scaling the digit arterial pressure waveform data with the selected scale. The method comprises uncentering, using the computational processing system, the scaled digit arterial pressure waveform data by adding an arterial pressure of the digit arterial pressure waveform data. The method further comprises, using the computational processing system, adding the selected offset to the uncentered and scaled digit arterial pressure waveform data.

In some implementations, the computational processing system is housed within a hemodynamic monitoring system.

In some implementations, the arterial pressure is one of: a mean arterial pressure, a systolic pressure, or a diastolic pressure.

In some implementations, the selecting step further comprises: selecting, using the computational processing system, the scale and the offset parameters via a lookup table comprising learned associations between feature vectors and function parameters of scale and offset.

In some implementations, the method further comprises training, using the computational processing system, a computational model to generate the lookup table. The training comprises learning associations between a cohort of patient data and radial intra-arterial pressure measurements captured via catheterization. The patient data comprises hemodynamic data obtained from a pressurized digit cuff and physiological data obtained from a photoplethysmogram.

In some implementations, the selecting step further comprises: selecting, using the computational processing system, the scale and the offset using a regression-based model or a classification-based model.

In some implementations, the method further comprises training, using the computational processing system, the regression-based model or the classification-based model by learning associations between cohort of patient data and radial intra-arterial pressure measurements captured via catheterization. The patient data comprises hemodynamic data obtained from a pressurized digit cuff and physiological data obtained from a photoplethysmogram.

In some implementations, the method further comprises

In some implementations, displaying, using the computational processing system, the radial arterial pressure waveform data on a display screen.

In some implementations, a hemodynamic monitoring system is for monitoring radial arterial pressure via captured digit arterial pressure. The system comprises a pressurized digit cuff, a photoplethysmogram, and a computational processing system in digital connection with the digit cuff and the photoplethysmogram. The computational processing system comprises a processor system and a memory system comprising one or more applications.

In some implementations, the one or more applications can direct the processor system to receive hemodynamic data derived from the pressurized digit cuff and physiological data derived from the photoplethysmogram. The hemodynamic data comprise digit arterial pressure waveform data and the physiological data are physiological information of the digit.

In some implementations, the one or more applications can direct the processor system to map the hemodynamic data and physiological data to a vector representative of the hemodynamic data and physiological data.

In some implementations, he one or more applications can direct the processor system to select a set of transformation function parameters based on the representative vector.

In some implementations, the one or more applications can direct the processor system to transform the digit arterial pressure waveform data into radial arterial pressure waveform data utilizing the selected set of transformation function parameters.

In some implementations, the one or more applications can direct the processor system to determine the digit arterial pressure data via a volume clamp method.

In some implementations, the hemodynamic data and physiological data further comprise one or more of: cardiac output, stroke volume, stroke volume variation, vascular tone, contractility, afterload, systemic vascular resistance, systolic pressure of a digit, diastolic pressure of a digit, mean arterial pressure of a digit, left ventricular ejection time, reconstructed brachial arterial waveform data, pulse pressure, DC, AC,

$$\frac{HR}{fSys} , \frac{fDia}{fMAP} , \frac{fSys - fDia}{fMAP} , \frac{fMAP - fDia}{fSys - fDia} , \frac{dP}{dt} , \frac{AC}{DC} ,$$

or instrumentation data comprising one or more: cuff type, cuff size, or sensor position in relation to heart.

In some implementations, the one or more applications can further direct the processor system to determine that a particular data point of the hemodynamic data and physiological data is beyond a limit threshold. The mapping step utilizes a limit value instead of the particular data point.

In some implementations, the mapping step comprises one of the following techniques: principal component analysis (PCA), kernel-PCA, graph-based kernel PCA, the Nyström method, linear discriminant analysis (LDA), generalized discriminant analysis (GDA), T-distributed stochastic neighbor embedding (t-SNE), or uniform manifold approximation and projection (UMAP).

In some implementations, the memory system further comprises a lookup table comprising learned associations between feature vectors and transformation function parameters. The one or more applications can further direct the processor system to select the set of transformation function parameters via the lookup table.

In some implementations, the lookup table had been generated via a trained computational model that has been trained on a cohort of patient data comprising hemodynamic data obtained from a pressurized digit cuff, physiological data obtained from a photoplethysmogram, and radial intra-arterial pressure measurements captured via catheterization.

In some implementations, the memory system further comprises a regression-based model or a classification-based model. The one or more applications can further direct the processor system to select the set of transformation function parameters using the regression-based model or the classification-based model.

In some implementations, the regression-based model or the classification-based model has been trained on a cohort of patient data comprising hemodynamic data obtained from a pressurized digit cuff, physiological data obtained from a photoplethysmogram, and radial intra-arterial pressure measurements captured via catheterization.

In some implementations, the one or more applications can further direct the processor system to determine that a particular parameter of the selected set of transformation function parameters is beyond a limit threshold. The one or more applications can further direct the processor system to select an alternative parameter value to replace the particular parameter that is beyond the limit threshold.

In some implementations, the alternative parameter value is one of: an upper limit value or a lower limit value.

In some implementations, the one or more applications can further direct the processor system to determine that the representative vector is abnormal. The selected set of transformation function parameters is one of: a previously selected set of transformation function parameters, a set of transformation function parameters having a set of prespecified values, or a combination of previously selected sets of transformation function parameters.

In some implementations, the transformation is performed by a linear transformation and the selected transformation function parameters are a scale and an offset. The one or more applications can further direct the processor system to center the digit arterial pressure waveform data by subtracting an arterial pressure of the digit arterial pressure waveform data. The one or more applications can further direct the processor system to scale the digit arterial pressure waveform data with the selected scale. The one or more applications can further direct the processor system to uncenter the scaled digit arterial pressure waveform data by adding the arterial pressure of the digit arterial pressure waveform data. The one or more applications can further direct the processor system to add the selected offset to the uncentered and scaled digit arterial pressure waveform data.

In some implementations, the arterial pressure is one of: a mean arterial pressure, a systolic arterial pressure, or a diastolic arterial pressure.

In some implementations, the hemodynamic monitoring system further comprises a display screen in connection with the processor system. The one or more applications can further direct the processor system to display the radial arterial pressure waveform data on a display screen.

In some implementations, a method is for training a model to predict a set of transformation function parameters for transforming digit arterial pressure data into radial arterial pressure data. The method comprises receiving, using a computational processing system, noninvasive hemodynamic data and physiological data and radial intra-arterial pressure waveform data from a cohort of patients. The method comprises clustering, using a computational processing system, similar data of the noninvasive hemodynamic data and physiological data to yield a plurality of clusters. The method comprises averaging, using a computational processing system, the similar data of each cluster. The method comprises mapping, using a computational processing system, the averaged data of each cluster to a feature vector representative of the noninvasive hemodynamic data and physiological data of each cluster. The method comprises, using a computational processing system, associating the representative feature vectors with the received radial intra-arterial pressure waveform data. The method comprises training, using a computational processing system, a computational model with the representative feature vectors and its associated radial intra-arterial pressure such that the computational model is trained to predict a set of transformation function parameters for transforming digit arterial pressure data into radial arterial pressure data.

In some implementations, the noninvasive digit hemodynamic data is derived from a finger cuff via a volume clamp method.

In some implementations, the noninvasive digit physiological data is derived from a photoplethysmogram.

In some implementations, the clustering step further comprises clustering, using the computational processing system, the similar hemodynamic data by connectivity-based clustering, centroid-based clustering, distribution-based clustering, density-based clustering, grid-based clustering, k-means clustering, or the Louvain method for community detection.

In some implementations, the regression-based model comprises LASSO regression, ridge regression, K-nearest neighbors, elastic net, least angle regression (LAR), or random forest regression.

In some implementations, the classification-based model comprises support vector machine (SVM), decision trees, random forests, or naïve Bayes.

In some implementations, the method further comprises generating, using the computational processing system, a lookup table of associations between features vectors and predicted sets of transformation function parameters.

### BRIEF DESCRIPTION OF THE DRAWINGS

The description and claims will be more fully understood with reference to the following figures and data graphs, which are presented as examples of the disclosure and should not be construed as a complete recitation of the scope of the disclosure.

FIGS. 1A to 1C provide examples of comparing generated waveforms with intra-arterial radial pressure waveforms. The panel in FIG. 1A depicts finger arterial pressure waveforms, the panel in FIG. 1B depicts reconstructed brachial arterial pressure waveforms, and the panel in FIG. 1C depicts reconstructed radial arterial pressure waveforms in accordance with methods and processes as described herein.

FIG. 2 provides an example of a method for reconstructing radial arterial pressure from digit arterial pressure.

FIG. 3 provides a conceptual illustration of transforming a digit radial arterial pressure waveform into a radial arterial pressure waveform.

FIG. 4 provides an example of a computational process for reconstructing radial arterial pressure from digit arterial pressure.

FIG. 5 provides a conceptual illustration of transforming a digit radial arterial pressure waveform into a radial arterial pressure waveform via (1) centering, (2) scaling, (3) uncentering and offsetting.

FIG. 6 provides an example of a method for training a computational model to predict transformation function parameters for transforming digit radial arterial pressure data into a radial arterial pressure data.

FIGS. 7A to 7C provide an example of clustering hemodynamic data for training a computational model to predict scale and offset.

FIG. 8 provides a conceptual illustration of a computational processing system for reconstructing radial arterial pressure data.

FIG. 9 provides a conceptual illustration of a hemodynamic monitoring system.

### DETAILED DESCRIPTION

The current disclosure details systems and methods to dynamically reconstruct radial arterial pressure from a continuous non-invasive digit arterial blood pressure measurement (e.g., measurements of a finger or a thumb). A transformation (e.g., linear transformation comprising scale and offset) can be determined by a trained computational model utilizing collected hemodynamic data and photoplethysmogram (PPG) data as input. Accordingly, a blood pressure monitoring system can comprise a digit cuff in connection with a computational system such that arterial pressure in the digit is recorded and the radial pressure is computed in real time.

Volume clamp methods of the prior art typically reconstructed brachial arterial pressure. In a surgical setting, however, a blood pressure waveform is usually only available when using an indwelling catheter within the radial artery. Converting the finger blood pressure to a radial arterial pressure will help the acceptance of finger pressure derived blood pressure waveforms in these settings. Many current methodologies rely on reconstructed brachial arterial pressure. Due to tapering of the arterial system towards the periphery, waveform distortion and some pressure fall is usually observed between brachial and radial measurement site. Brachial arterial pressure typically overestimates radial mean arterial pressure (MAP) and radial diastolic pressure (DP) and underestimates radial dP/dt (maximum slope of the blood pressure waveform during systole). For accurate prediction of hypotension and derived hemodynamic parameters such as cardiac output, overestimated biases in MAP, DBP and dP/dt needs to be avoided. Accordingly, there is a need for more accurate and direct reconstruction of radial arterial pressure.

A common issue with volume clamp methods is that the measured blood pressure waveform is affected by vasoconstriction in the finger arteries, which could lead to various issues including underestimation of the pulse pressure. Accordingly, there is a need to better estimate pulse pressure when patients are experiencing vasoconstriction in their finger arteries.

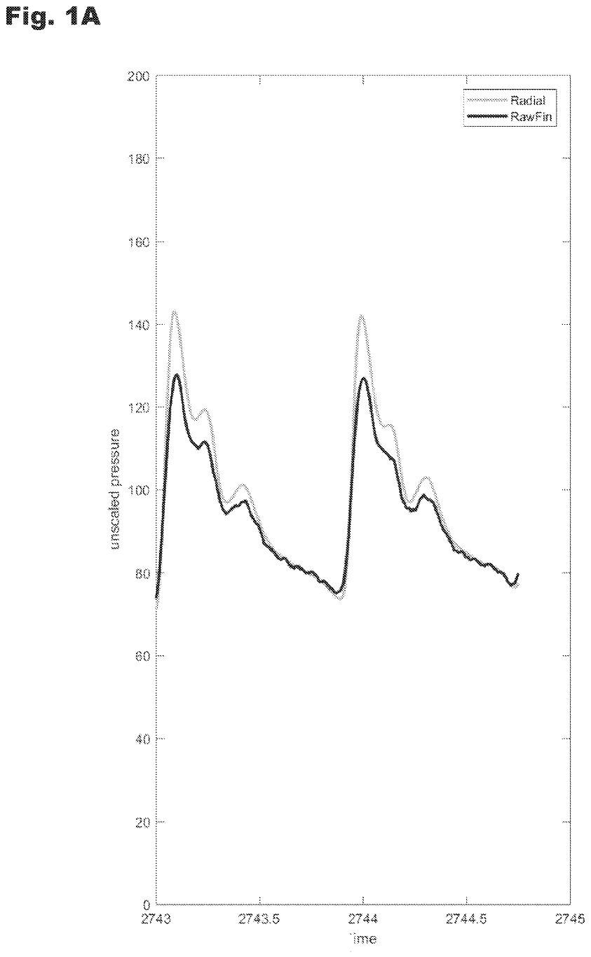

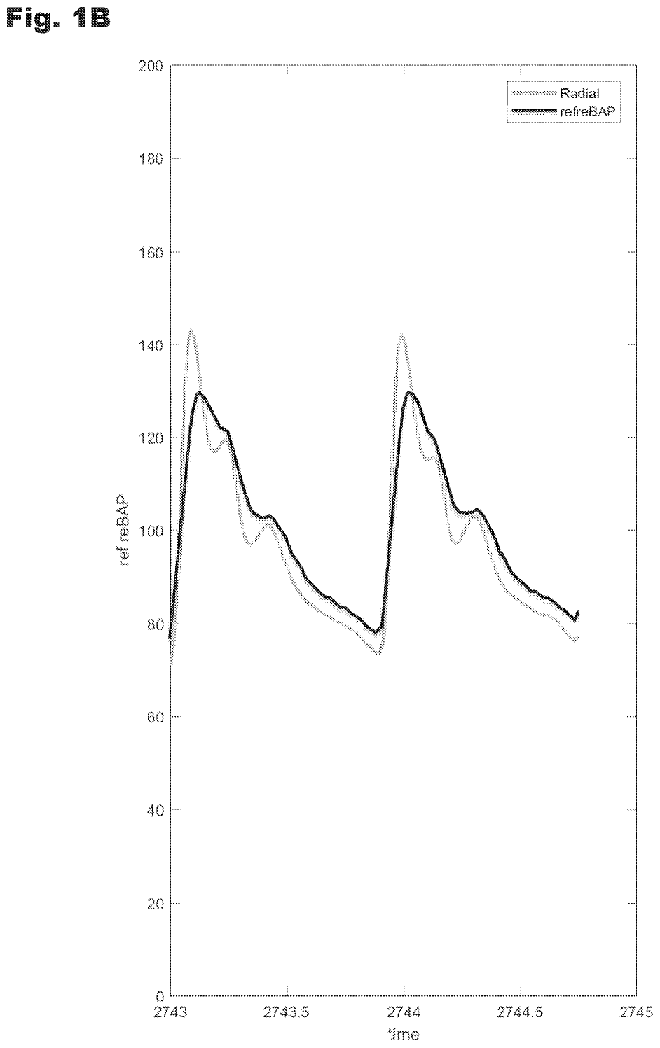

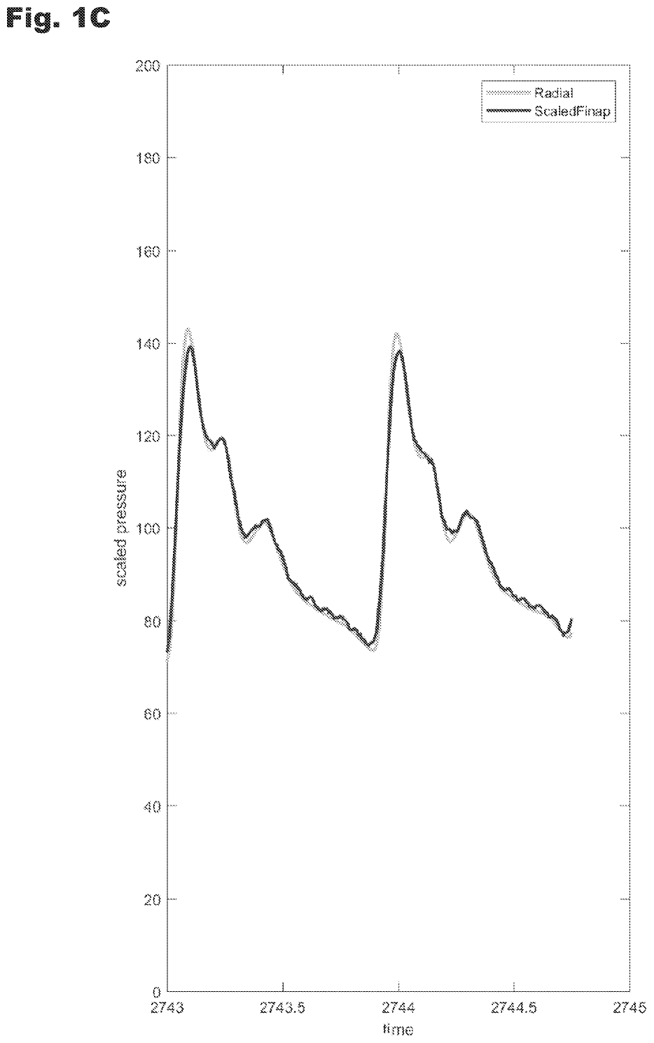

Here, novel systems and methods to reconstruct radial arterial pressure via volume clamp determination of digit arterial pressure are described. The various systems and methods described herein improve the ability to reconstruct radial arterial pressure. Provided in FIG. 1 are exemplary reconstructions of radial arterial pressure waveforms. In each of the three waveforms shown, the grey solid line waveform is intra-radial arterial pressure as measured via catheterization. In the left panel, the dark solid line waveform is finger arterial pressure as measured by the volume clamp method. In the middle panel, the dark solid line waveform is reconstructed brachial arterial pressure, as reconstructed by methods of the prior art. In the right panel, the dark solid waveform is reconstructed radial arterial pressure via systems and methods of the current disclosure. As can be readily determined, the reconstructed radial arterial pressure provides the best estimate of true intra-radial arterial pressure.

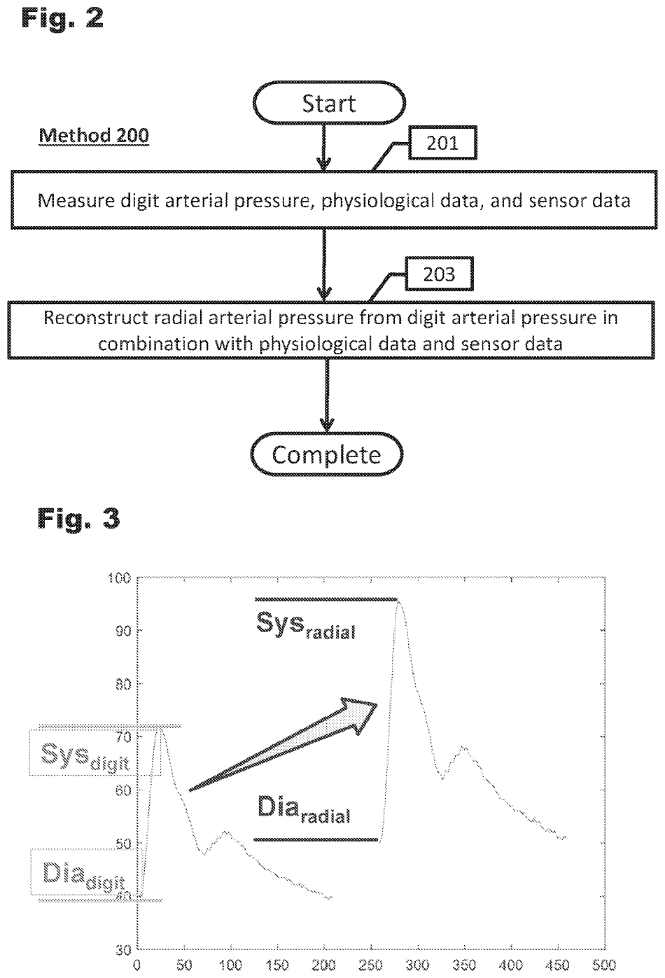

An example of a method for reconstructing radial arterial pressure is provided in FIG. 2. Method 200 measures (201) digit arterial pressure, physiological data, and sensor data. Any digit can be utilized (finger or thumb) and any means for measuring digit arterial pressure can be utilized. One common method of measuring digit arterial pressure is the volume clamp method, utilizing a pressurized digit cuff, a light source, and a light sensor. Physiological data can include physiological information of the digit captured via a photoplethysmogram, such as (for example) arterial volume. Sensor data is data related to the instrumentation for collecting arterial pressure data and physiological data.

Method 200 also reconstructs (203) radial arterial pressure from digit arterial pressure in combination with physiological data, and sensor data. To reconstruct radial arterial pressure, a transformation can be utilized that scales and offsets the measured digit arterial pressure. The digit arterial blood pressure waveform, obtained via volume clamp method, is sufficiently similar in waveform shape to the radial arterial waveform, and therefore a linear scaling can be applied. An example of a linear transformation is provided in the following equation:

$${Pressure}_{radial} = offset + scale \times {pressure}_{digit}$$

The equation can be utilized to transform digit arterial pressure waveforms to radial arterial pressure waveforms. Provided in FIG. 3 is a conceptual example of digit waveform transformed into a radial pressure waveform, which has been rescaled and offset.

In accordance with various methods of the disclosure, the function to transfer digit pressure to a more proximal pressure waveform, such as radial arterial pressure, is dependent on a number of parameters. These parameters can include (but are not limited to) hemodynamic data from the digit waveform, hemodynamic data from a reconstructed proximal waveform, physiological information including vascular state from photoplethysmography, patient data, and data related to the instrumentation.

Since the transfer between digit pressure and radial pressure can be described by a linear function, this disclosure will focus on the derivation of scale and offset. However, other implementations may use more complex non-linear functions. Accordingly, computational models can be trained to determine parameters for non-linear functions in essentially the same methodologies described herein for linear transformation.

To determine linear transformation, a computational model can be trained on hemodynamic data, physiological data, and sensor data that was collected from a cohort of patients. The patient hemodynamic data, physiological data, and sensor data can be associated with patient radial intra-arterial pressure that was simultaneously collected. Because the collected hemodynamic data, physiological data, and sensor data can be vast in size, in some implementations, the trained computational model utilizes a data reducing clustering technique such that high volumes of data can be utilized to train the model.

While specific examples of methods to reconstruct radial arterial pressure are described above, one of ordinary skill in the art can appreciate that various steps of the method can be performed in different orders and that certain steps may be optional according to various implementations. As such, it should be clear that the various steps of the method could be used as appropriate to the requirements of specific applications. Furthermore, any of a variety of methods to reconstruct radial arterial pressure appropriate to the requirements of a given application can be utilized in various implementations.

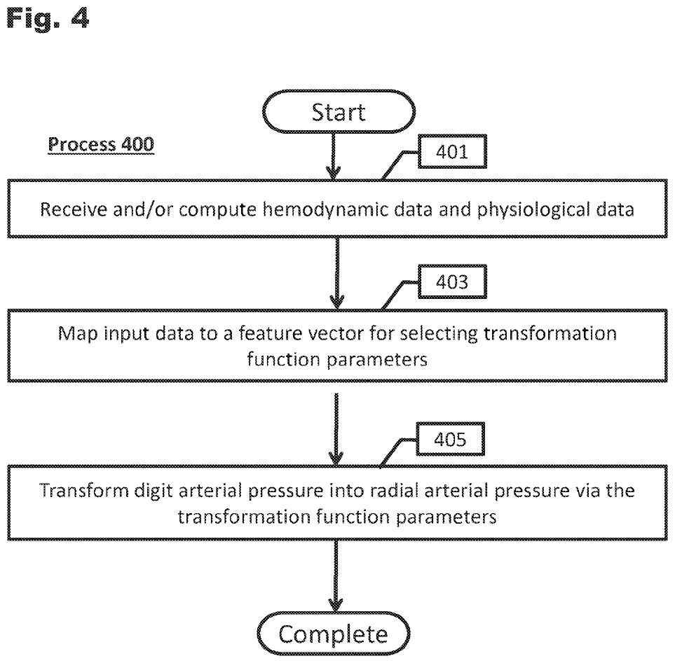

Several methods are directed towards computationally reconstructing radial arterial pressure from a set of hemodynamic measurements and physiological measurements recorded in a digit in real time. Generally, a computational processing system can receive hemodynamic data and physiological data that is inclusive of arterial pressure measured in a digit. The hemodynamic dynamic data and physiological data can be processed by the computational system to determine parameters for function parameters. The function parameters are used to transform the digit arterial pressure waveforms into a radial arterial pressure waveform. Provided in FIG. 4 is an example of a process to be performed by a computational processing system that yields reconstructed radial arterial pressure from arterial pressure captured in a digit. Process 400 begins by receiving and/or computing (401) hemodynamic data and physiological data. The hemodynamic data and physiological to be received includes data captured via a digit cuff and a photoplethysmogram. In some instances, the hemodynamic data and physiological to be received includes computed data derived from the captured data. The hemodynamic data to be received comprises the arterial waveform data captured via the digit cuff, which will be utilized to reconstruct a radial arterial waveform. Any appropriate method to measure pressure waveform can be utilized, such as (for example) the volume clamp method. Other hemodynamic data to be received and/or computed can further comprise at least one of: cardiac output, stroke volume, stroke volume variation, vascular tone, contractility, afterload, systemic vascular resistance, systolic pressure of a digit, diastolic pressure of a digit, mean arterial pressure of a digit, left ventricular ejection time, and reconstructed brachial arterial waveform data. Physiological data to be received and/or computed can comprise at least one of: pulse pressure, DC (PPG signal attributed to absorption of skin tissue), and AC (PPG signal attributed to arterial blood). In some implementations, one or more data ratios are received and/or computed. Data ratios to be received and/or computed can comprise at least one of:

$$\frac{HR}{fSys} , \frac{fDia}{fMAP} , \frac{fSys - fDia}{fMAP} , \frac{fMAP - fDia}{fSys - fDia} , \frac{dP}{dt} , and \frac{AC}{DC} .$$

In some implementations, instrumentation data are to be received. Instrumentation data can comprise at least one of: cuff type, cuff size, and sensor position in relation to heart. Cuff type can refer to a particular digit utilized to capture data or other particularities related to the pressurized digit cuff.

Digit arterial pressure waveform data can be assessed at any appropriate sampling frequency. The hemodynamic and the physiological data can be averaged data over a period of time. In various implementations, the hemodynamic and the physiological data can be averaged (for example) over 10 seconds, over 20 seconds, over 30 seconds, over 40 seconds, over 50 seconds, or over 60 seconds.

In some implementations, the range of the hemodynamic data or of the physiological data is limited. If a particular data point is beyond a limit threshold, a limit value is utilized instead of the particular hemodynamic data point. For instance, if the hemodynamic data point is above the upper limit threshold, then the upper limit value is utilized. Likewise, if the hemodynamic data point is below the lower limit threshold, then the lower limit value is utilized.

Process 400 can map (403) the input data to a feature vector for selecting transformation function parameters. In some implementations, the received and/or computed hemodynamic data and physiological data are entered as parameters in computational model to yield a formula to transform the digit arterial pressure into a reconstructed radial arterial pressure.

To yield a feature vector, Process 400 can perform dimensionality expansion on the hemodynamic data features to yield a feature vector. The feature vector yielded is representative of the current hemodynamics occurring in the patient in real time and is utilized for selecting a scale and an offset to reconstruct a radial waveform. Any appropriate technique to map inputs to a feature vector can be utilized, including (but not limited to) principal component analysis (PCA), kernel methods such as kernel-PCA, graph-based kernel PCA, the Nyström method. These mappings can be combined with dimension reduction methods, such linear discriminant analysis (LDA), generalized discriminant analysis (GDA), T-distributed stochastic neighbor embedding (t-SNE), and uniform manifold approximation and projection (UMAP).

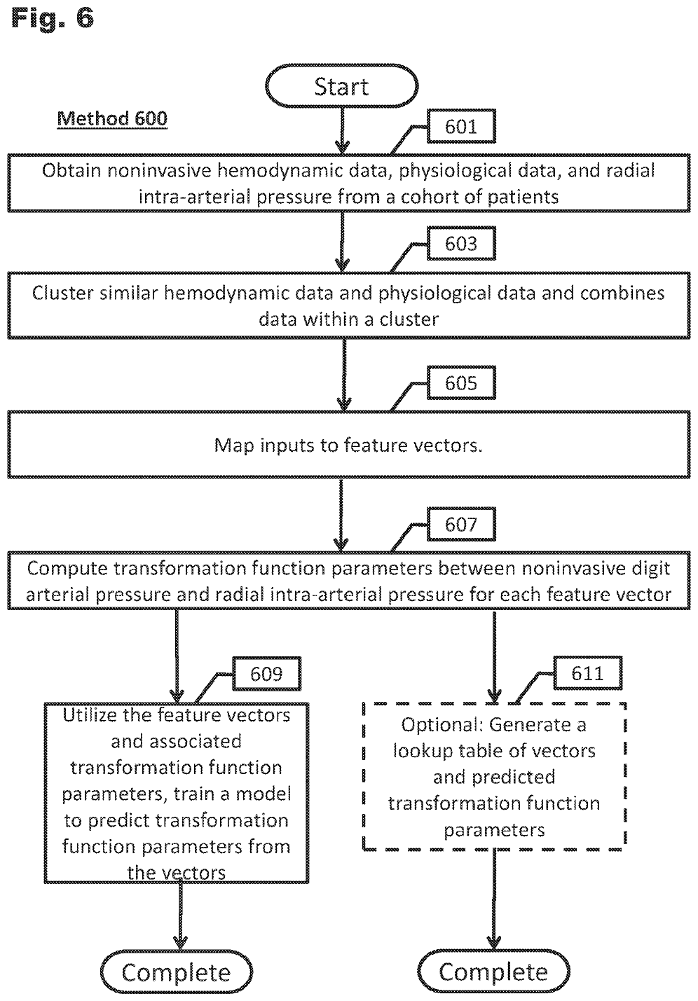

The vector is utilized to select transformation function parameters. In some implementations, a linear transformation is performed and scale and offset are selected. Any appropriate technique for selecting transformation function parameters can be utilized. In some implementations, the vector is utilized with a lookup table that has saved associations between vectors and the transformation function parameters. These associations can be implemented with a computational model that is trained on patient data inclusive of the hemodynamic data and physiological data derived from a digit cuff and radial intra-arterial pressure measurements. Radial intra-arterial pressure measurements can be captured via catheterization. For more on model training, see FIG. 6 and associated description.

In some implementations, the vector is entered into a trained computational model to select transformation function parameters. The trained computational model can learn associations between vectors and transformation function parameters. Any appropriate model architecture can be utilized. Various models can be utilized, including (but not limited to) regression-based or classification-based models. Regression-based models include (but are not limited to) LASSO regression, ridge regression, k-nearest neighbors, elastic net, least angle regression (LAR), and random forest regression. Classification-based models include (but are not limited to) support vector machines (SVMs), decision trees, random forests, and naïve Bayes. In some implementations, the model is regularized.

The function parameters available for selection via any method can be limited. That is, when a vector yields selection of a function parameter that is beyond a limit threshold, such as an upper limit threshold or a lower limit threshold, an alternative value is selected instead. In some implementations, an alternative value selected is an upper limit value is selected or a lower limit value is selected. When a lookup table is utilized, the function parameters can be limited within the table.

When mapping the inputs to a feature vector, if the vector is abnormal, the process can instead select alternative function parameters. In various implementations, the alternative function parameters can be: previously selected function parameters, the immediately preceding function parameters selected, a prespecified value of function parameters (e.g., default function parameters), or a combination of previously selected function parameters (e.g., an averaged amount of previously selected function parameters). Abnormality of a vector can be determined by any means. When utilizing a lookup table, the table can include only acceptable vectors and thus when a vector does not match any vectors in the lookup table, alternative function parameters are selected. Alternatively, a lookup table can include abnormal vectors that when selected results in selection of alternative function parameters.

In some implementations, the transitions between waveforms is smoothened. Smoothing can be implemented with a low-pass filter on the instantaneously selected function parameters. Smoothing out transitions can prevent discontinuities in output.

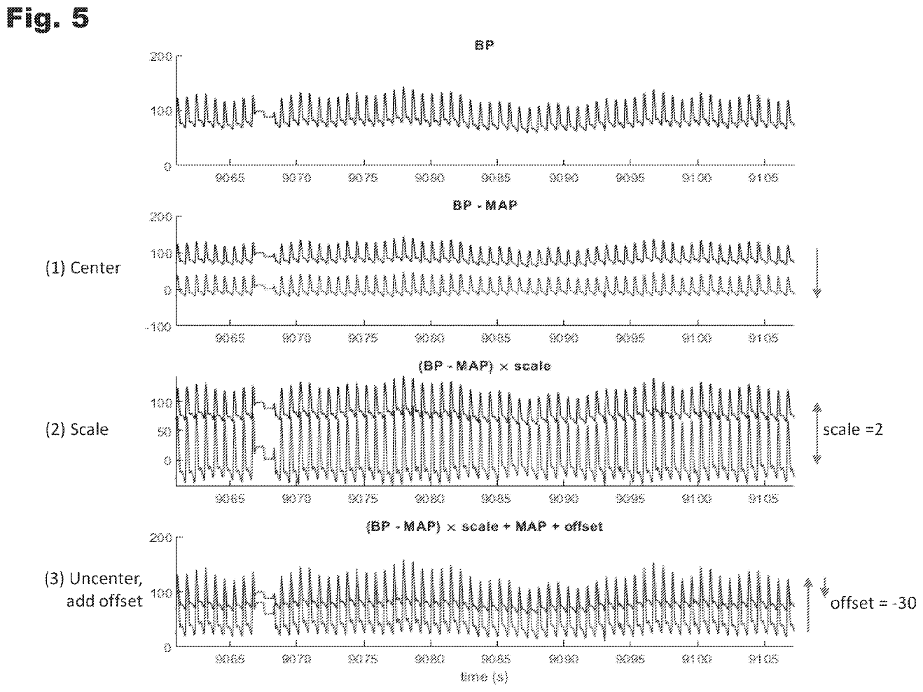

Once a scale and offset are selected, process 400 transforms (405) the digit arterial pressure into radial arterial pressure via the selected function parameters. In some implementations, linear transformation is performed with function parameters of scale and offset. In a particular example to reconstruct radial arterial pressure vial scale and offset, the digit arterial pressure waveform can be (1) centered by subtracting an arterial pressure such that the waveform is centered at 0, (2) the scale can be applied, and (3) the wave form can be uncentered by adding back the arterial pressure and the offset can be added. The arterial pressure that can be utilized for subtracting and adding back can be any measure or computed arterial pressure, including (but not limited to) MAP, systolic arterial pressure, and diastolic arterial pressure. Provided in FIG. 5 is an exemplary linear transformation in which the selected scale is 2 and the selected offset is −30.

The noninvasive reconstructed radial arterial pressure waveform can be presented on a patient monitor or utilized for other downstream applications. In some instances, the reconstructed radial arterial pressure is utilized to calculate a proximal arterial pressure, cardiac output, or other hemodynamic functions.

While specific examples of processes to reconstruct radial arterial pressure are described above, one of ordinary skill in the art can appreciate that various steps of the process can be performed in different orders and that certain steps may be optional according to various implementations. As such, it should be clear that the various steps of the process could be used as appropriate to the requirements of specific applications. Furthermore, any of a variety of processes to reconstruct radial arterial pressure appropriate to the requirements of a given application can be utilized in various implementations.

Various methods can be utilized to train a model to be utilized for determining function parameters for reconstruction radial arterial pressure. In some implementations, the trained model is utilized in real time to select function parameters from a patient's hemodynamic data and physiological data. In some implementations, the trained model is utilized to generate a lookup table in which the lookup table is utilized in real time to select a scale and an offset from a patient's hemodynamic data. Generally, a model can be trained from a cohort of individuals that have their noninvasive hemodynamic pressure data collected via a digit cuff and their radial intra-arterial pressure collected from catheter. The model can learn associations between the noninvasive hemodynamic pressure data and radial intra-arterial pressure such that it can predict scale and offset to reconstruction radial arterial pressure from the noninvasive hemodynamic pressure data.

Provided in FIG. 6 is an example of a method for training a computational model to predict scale and offset from noninvasive hemodynamic data and physiological data obtained from a digit cuff and photoplethysmogram. Method 600 obtains (601) noninvasive hemodynamic data, physiological data, and radial intra-arterial pressure from a cohort of patients. The noninvasive hemodynamic data can be collected via a digit cuff by the volume clamp method. The radial intra-arterial pressure can be collected via an invasive means that provides accurate intra-arterial pressure (e.g., catheterization). For example, a catheter with a pressure sensor can be inserted into the radial artery. The data can be derived from any cohort of individuals. Many medical procedures have been performed utilizing a noninvasive hemodynamic data obtained from a digit cuff, physiological data obtained from a photoplethysmogram, and a catheter in the radial artery and thus data derived from these medical procedures can be obtained. In some instances, the hemodynamic data and physiological data further include computed data derived from the captured data.

The noninvasive hemodynamic data can comprise digit arterial waveforms, cardiac output, stroke volume, stroke volume variation, vascular tone, contractility, afterload, systemic vascular resistance, systolic pressure of a digit, diastolic pressure of a digit, mean arterial pressure of a digit, left ventricular ejection time, reconstructed brachial arterial waveform data,

$$\frac{HR}{fSys} , \frac{fDia}{fMAP} , \frac{fSys - fDia}{fMAP} , \frac{fMAP - fDia}{fSys - fDia} , and \frac{dP}{dt} .$$

Physiological data can comprise pulse pressure, DC (PPG signal attributed to absorption of skin tissue), AC (PPG signal) attributed to arterial blood), and

$$\frac{AC}{DC} .$$

Instrumentation data can also be obtained, and the instrumentation data can comprise cuff type, cuff size, and sensor position in relation to heart.

To manage the large amount of data points, Method 600 clusters (603) similar hemodynamic data and physiological data and combines the data within a cluster. Data can be combined by any statistical method to combine data, such as (for example) averaging the data. Further, any appropriate clustering technique can be utilized, including (but not limited to) connectivity-based clustering, centroid-based clustering, distribution-based clustering, density-based clustering, grid-based clustering, k-means clustering, and the Louvain method for community detection.

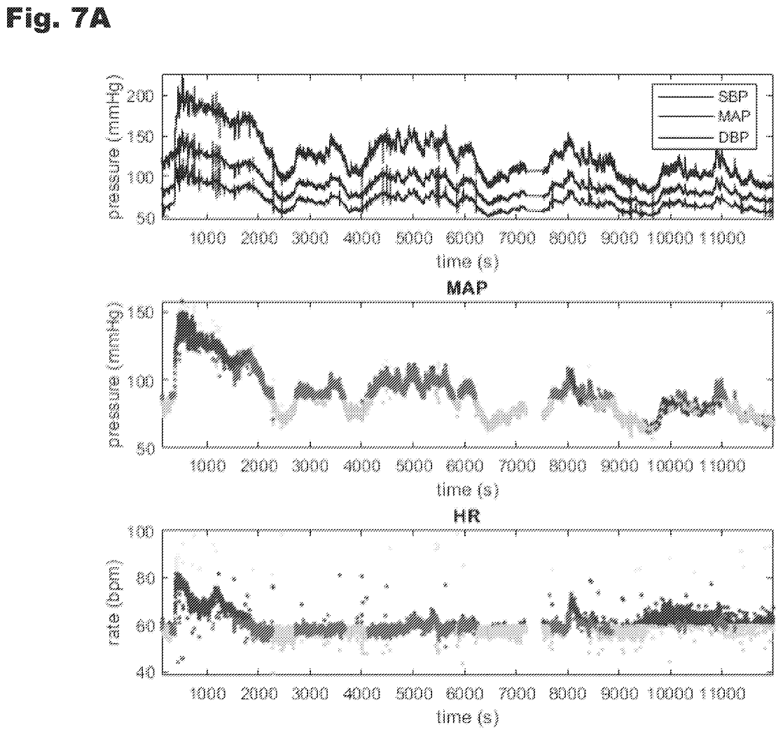

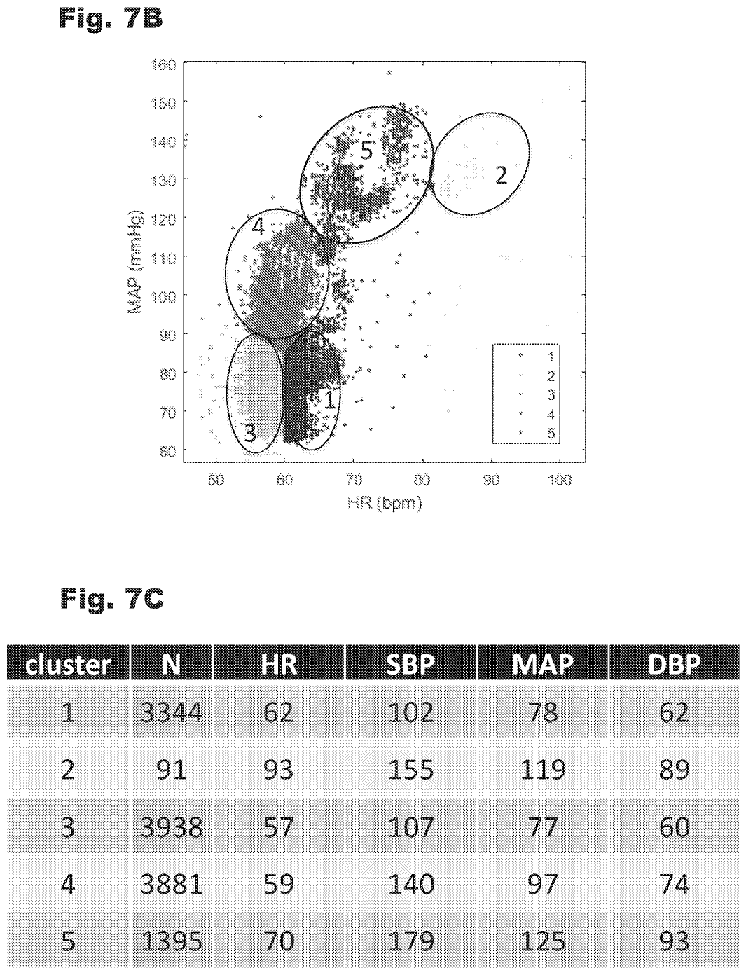

Provided in FIGS. 7A to 7C is an example of a small collection of timepoint hemodynamic data that is clustered. FIG. 7A shows systolic blood pressure, mean arterial blood pressure, diastolic blood pressure, and heart rate captured over time. The timepoints can be clustered based on similarity of hemodynamic data (FIG. 7B, clusters are encircled). The data within timepoint that are clustered can then be averaged to perform data reduction and model training. Clustering of similar data reduces the representation of common data and increases the representation data of uncommon data (FIG. 7C). For instance, cluster #2 has 91 data points and cluster #3 has 3938 datapoints. By averaging the data within the cluster, and then using the averaged data to build a model, cluster #2 will have the same impact as cluster #3. This can help the model better learn and interpret the less common timepoints of hemodynamic data.

The average hemodynamic data and physiological data of each cluster is mapped (605) to vectors to generate a collection of feature vectors. Any appropriate technique to map inputs to features can be utilized, including (but not limited to) principal component analysis (PCA), non-negative matrix factorization (NMF), kernel PCA, graph-based kernel PCA, the Nyström method, linear discriminant analysis (LDA), generalized discriminant analysis (GDA), T-distributed stochastic neighbor embedding (t-SNE), and uniform manifold approximation and projection (UMAP).

For each feature vector, transformation function parameters are each computed (607) between the noninvasive digit arterial pressure and the radial intra-arterial pressure. In some implementations, the transformation function is linear and the transformation function parameters arc scale and offset. The feature vectors and associated transformation function parameters arc utilized (609) to train a model to predict transformation function parameters from the feature vectors. Any appropriate model architecture can be utilized, including (but not limited to) regression-based or classification-based models. Regression-based models include (but are not limited to) LASSO regression, ridge regression, K-nearest neighbors, elastic net, least angle regression (LAR), and random forest regression. Classification-based models include (but are not limited to) support vector machine (SVM), decision trees, random forests, and naïve Bayes. In some implementations, the model is regularized.

Once a model is trained, it can be utilized to predict transformation function parameters at each timepoint using the hemodynamic data and physiological data at that timepoint. Accordingly, the trained model can be utilized in association with a noninvasive pressurized digit cuff and photoplethysmogram to predict transformation function parameters such that the digit arterial pressure can be transformed into a radial arterial pressure.

In some implementations, Method 600 generates (611) a lookup table of vectors and predicted transformation function parameters. Accordingly, the lookup table can be utilized in association with a noninvasive pressurized digit cuff and photoplethysmogram to predict transformation function parameters such that the digit arterial pressure can be transformed into a radial arterial pressure.

In some implementations, a clustering method is utilized to construct a lookup table for scale and offset parameters. Clusters can be generated and transformation function parameters can be assigned to particular clusters to learn an association. When a vector associates with a particular cluster, the assigned transformation function parameters to that cluster can be utilized. Clustering methods include (but are not limited to) connectivity-based clustering, centroid-based clustering, distribution-based clustering, density-based clustering, grid-based clustering, k-means clustering, and the Louvain method for community detection.

While specific examples of methods to train a computational model to predict transformation function parameters are described above, one of ordinary skill in the art can appreciate that various steps of the process can be performed in different orders and that certain steps may be optional according to various implementations. As such, it should be clear that the various steps of the process could be used as appropriate to the requirements of specific applications. Furthermore, any of a variety of processes to train a computational model to predict transformation function parameters appropriate to the requirements of a given application can be utilized in various implementations.

### Computational Processing System

A computational processing system to reconstruct radial arterial pressure in accordance with the various methods and processes of the disclosure typically utilizes a processing system including one or more of a CPU, GPU and/or neural processing engine. As described herein, digit arterial pressure can be recorded and transformed into radial arterial pressure in real time using a computational processing system.

The computational processing system can be housed within a patient monitor in a direct wired connection between the monitor and components, inclusive of a pressurized digit cuff, photoplethysmogram and a heart position reference system. Alternatively, the computational processing system can be housed separately from the patient monitor and components, receiving the acquired digit arterial pressure via a wireless connection (e.g., WiFi, cellular, Bluetooth, etc). The computational processing system can be implemented on any appropriate computing device such as (but not limited to) a tablet and/or portable computer.

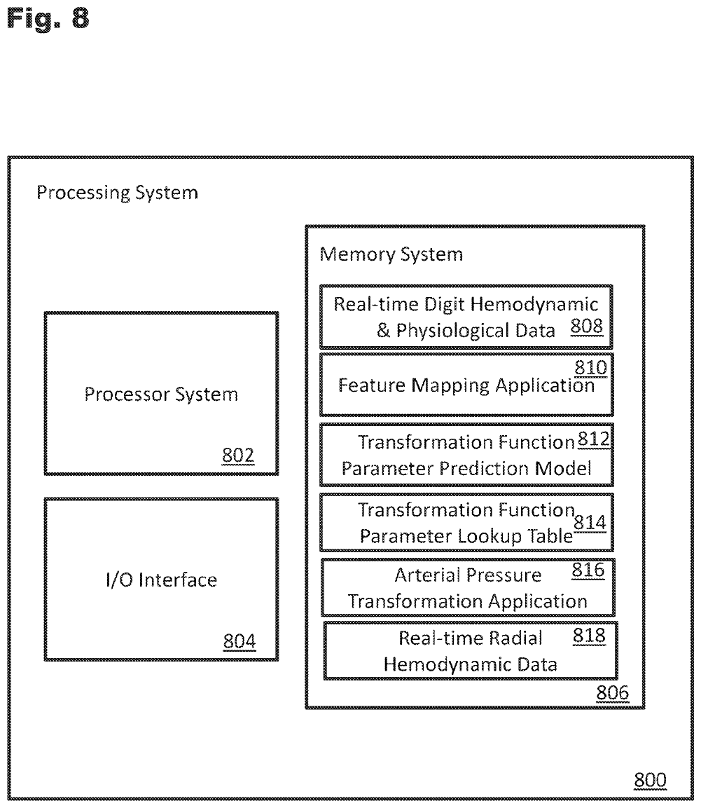

An exemplary computational processing system that can be utilized to perform the various methods and processes of the disclosure is illustrated in FIG. 8. The computational processing system 800 includes a processor system 802, an I/O interface 804, and a memory system 806. As can readily be appreciated, the processor system 802, I/O interface 804, and memory system 806 can be implemented using any of a variety of components appropriate to the requirements of specific applications including (but not limited to) CPUs, GPUs, ISPs, DSPs, wireless modems (e.g., WiFi, Bluetooth modems), serial interfaces, volatile memory (e.g., DRAM) and/or non-volatile memory (e.g., SRAM, and/or NAND Flash).

In the illustrated example, the memory system is capable of storing various data, applications, and models. It is to be understood that the listed data, applications and models are a representative sample of what can be stored in memory and that various memory systems may store some or all of the various data, applications, and models listed. Further, any combination of data, applications, and models can be stored, and in some implementations, various data, applications, and/or models are stored temporarily.

In some implementations, the memory system 806 can store real-time digit hemodynamic and physiological data 808, which can be obtained from a pressurized digit finger cuff via the volume clamp method and from a photoplethysmogram. A feature mapping application 810 can be stored in the memory system 806 utilize real-time digit hemodynamic and physiological data 808 to generate vectors. The generated vectors can be utilized in a transformation function parameter prediction model 812 and/or a transformation function parameter lookup table 814, each of which can be stored in the memory system 806, to determine appropriate transformation function parameters. An arterial pressure transformation application 816, which can be stored in the memory system 806, can utilize the selected transformation function parameters to transform the real-time digit hemodynamic data 808 into real-time radial hemodynamic data 818, which can be stored in the memory system 806. Further, the real-time digit hemodynamic data 808 and/or the real-time radial hemodynamic data 818 can be displayed on a monitor other screen via the I/O interface 804.

While specific computational processing systems are described above with reference to FIG. 8, it should be readily appreciated that computational processes and/or other processes utilized in the provision of radial arterial pressure reconstruction can be implemented on any of a variety of processing devices including combinations of processing devices. Accordingly, computational devices should be understood as not limited to specific monitoring systems, computational processing systems, and/or specific applications and models. Computational devices can be implemented using any of the combinations of systems described herein and/or modified versions of the systems described herein to perform the processes, combinations of processes, and/or modified versions of the processes described herein.

### Hemodynamic Monitoring System

The systems and methods of the current disclosure can be utilized within a noninvasive hemodynamic monitoring system. Generally, the noninvasive hemodynamic monitoring system includes a pressurized cuff, a photoplethysmograph, a pressure regulator, and a computational system. Provided in FIG. 1 is an example of a noninvasive hemodynamic monitoring system 900 as would be utilized on a digit of an individual. On the digit is a blood pressure cuff with PPG 902 that keeps the artery within the digit at a constant diameter when performing arterial pressure measurements. The pressure in the cuff represents the arterial pressure within the digit. The PPG can further provide physiological data.

The blood pressure cuff with PPG can be in connection with hemodynamic monitoring system 900 and pressure pump system 904. Data can be transmitted between the PPG 902 and hemodynamic monitoring system. Pump system 904 provides pressure to the blood pressure cuff. Pump system 904 can further be in connection with the hemodynamic monitoring system 900 such that the monitoring system can instruct the pump system to provide the requisite amount of pressure to cuff 902 to keep the artery diameter constant.

Hemodynamic monitoring system 900 can comprise a computational system, such as (for example) the system portrayed and described in reference to FIG. 8. Hemodynamic monitoring system 900 can comprise a processor system 906 for and I/O interface 908 for input and output of data, such as data communicated between hemodynamic monitoring system 900 with PPG 902, pump system 904, and a user interface. Hemodynamic monitoring system 900 can utilize a number of applications stored within a memory system 910 to be executed by processor system 906. Applications that can be stored within a memory system 910 include real-time hemodynamic data applications 912, calibration applications 914, and pressure regulation applications 916 for operating the hemodynamic monitoring system.

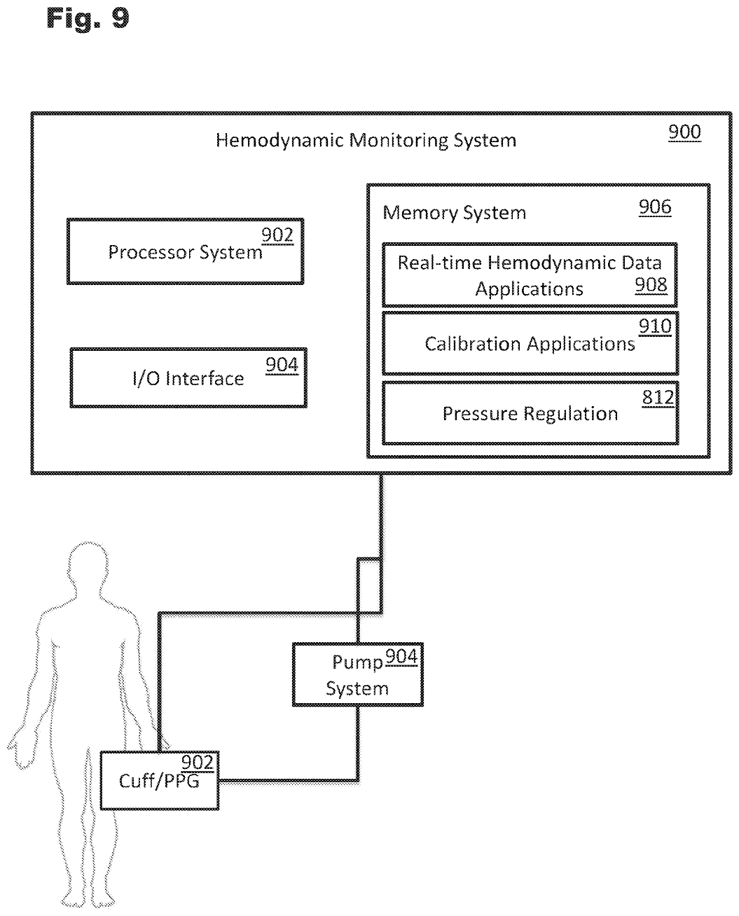

While a specific hemodynamic monitoring system configuration is described above with reference to FIG. 9, it should be readily appreciated that various hemodynamic monitoring systems and/or other medical monitoring utilized in the provision of hemodynamic monitoring can be implemented in any of a variety of configurations. Accordingly, the various systems and methods described herein should be understood as not to be limited to any specific hemodynamic monitoring system, but instead can be implemented using any variety of hemodynamic monitoring or medical monitoring systems capable of measuring arterial blood pressure within a digit.

### EXAMPLES

Example 1: A real-time method for selecting a set of transformation function parameters for transforming digit arterial pressure data into radial arterial pressure data, comprising:
receiving, using a computational processing system, hemodynamic data and physiological data, wherein the hemodynamic data and physiological data comprise sensor readings of a digit, wherein the hemodynamic data comprise digit arterial pressure waveform data and the physiological data are physiological information of the digit;
mapping, using the computational processing system, the hemodynamic data and physiological data to a vector representative of the hemodynamic data and physiological data; and
selecting, using the computational processing system, a set of transformation function parameters based on the representative vector, wherein the set of transformation parameters can be utilized to transform the digit arterial pressure waveform data into radial arterial pressure waveform data.
Example 2: The method of example 1, wherein the computational processing system is housed within a hemodynamic monitoring system.
Example 3: The method of example 2, wherein the hemodynamic monitoring system comprises a pressurized digit cuff, the method further comprising: obtaining a digit arterial pressure data via a volume clamp method.
Example 4: The method of example 2 or 3, wherein the hemodynamic monitoring system comprises a photoplethysmogram, the method further comprising: obtaining the sensor readings via the photoplethysmogram.
Example 5: The method of any one of examples 1 to 4, wherein the hemodynamic data and physiological data further comprise one or more of: cardiac output, stroke volume, stroke volume variation, vascular tone, contractility, afterload, systemic vascular resistance, systolic pressure of a digit, diastolic pressure of a digit, mean arterial pressure of a digit, left ventricular ejection time, reconstructed brachial arterial waveform data, pulse pressure, DC, AC,

$$\frac{HR}{fSys} , \frac{fDia}{fMAP} , \frac{fSys - fDia}{fMAP} , \frac{fMAP - fDia}{fSys - fDia} , \frac{dP}{dt} , \frac{AC}{DC} ,$$

or instrumentation data comprising one or more: cuff type, cuff size, or sensor position in relation to heart.
Example 6: The method of any one of examples 1 to 5 further comprising determining, using the computational processing system, that a particular data point of the hemodynamic data and physiological data is beyond a limit threshold; wherein the mapping step further comprises: utilizing a limit value instead of the particular data point.
Example 7: The method of any one of examples 1 to 6, wherein the mapping step comprises performing one of the following techniques: principal component analysis (PCA), kernel-PCA, graph-based kernel PCA, the Nyström method, linear discriminant analysis (LDA), generalized discriminant analysis (GDA), T-distributed stochastic neighbor embedding (t-SNE), or uniform manifold approximation and projection (UMAP).
Example 8: The method of any one of examples 1 to 7, wherein the selecting step further comprises: selecting, using the computational processing system, the set of transformation function parameters via a lookup table comprising learned associations between feature vectors and transformation function parameters.
Example 9: The method of example 8 further comprising: training, using the computational processing system, a computational model to generate the lookup table, wherein the training comprises learning associations between a cohort of patient data and radial intra-arterial pressure measurements captured via catheterization, wherein the patient data comprises hemodynamic data obtained from a pressurized digit cuff and physiological data obtained from a photoplethysmogram.
Example 10: The method of any one of examples 1 to 7, wherein the selecting step further comprises: selecting, using the computational processing system, the set of transformation function parameters using a regression-based model or a classification-based model.
Example 11: The method of example 10 further comprising: training, using the computational processing system, the regression-based model or the classification-based model by learning associations between cohort of patient data and radial intra-arterial pressure measurements captured via catheterization, wherein the patient data comprises hemodynamic data obtained from a pressurized digit cuff and physiological data obtained from photoplethysmogram.
Example 12: The method of any one of examples 1 to 11 further comprising:
determining, using the computational processing system, that a particular parameter of the selected set of transformation function parameters is beyond a limit threshold; and
selecting, using the computational processing system, an alternative parameter value to replace the particular parameter that is beyond the limit threshold.
Example 13: The method of example 12, wherein the alternative parameter value is one of: an upper limit value or a lower limit value.
Example 14: The method of any one of examples 1 to 13 further comprising determining, using the computational processing system, the representative vector is abnormal; and wherein the selecting step further comprises: selecting, using the computational processing system, one of: a previously selected set of transformation function parameters, a set of transformation function parameters having a set of prespecified values, or a combination of previously selected sets of transformation function parameters.
Example 15: The method of any one of examples 1 to 14 further comprising transforming, using the computational processing system, the digit arterial pressure waveform data into radial arterial pressure waveform data utilizing the selected set of transformation function parameters.
Example 16: The method of example 15, wherein the transforming step is performed by a linear transformation and the selected transformation function parameters are a scale and an offset, wherein the method further comprises:
centering, using the computational processing system, the digit arterial pressure waveform data by subtracting an arterial pressure of the digit arterial pressure waveform data;
scaling, using the computational processing system, the digit arterial pressure waveform data with the selected scale;
uncentering, using the computational processing system, the scaled digit arterial pressure waveform data by adding the arterial pressure of the digit arterial pressure waveform data; and
adding, using the computational processing system, the selected offset to the uncentered and scaled digit arterial pressure waveform data to yield the radial arterial pressure waveform data.
Example 17: The method of example 16, wherein the arterial pressure is one of: a mean arterial pressure, a systolic arterial pressure, or a diastolic arterial pressure.
Example 18: The method of example 15, 16, or 17 further comprising displaying, using the computational processing system, the radial arterial pressure waveform data on a display screen.
Example 19: The method of any one of examples 15 to 18 further comprising:
determining, using the computational processing system, proximal arterial pressure waveform data from the radial arterial pressure waveform data; and
displaying, using the computational processing system, the proximal arterial pressure waveform data on a display screen.
Example 20: A method of transforming digit arterial pressure waveform data into radial arterial pressure waveform data, comprising:
selecting, using a computational processing system, a scale and an offset;
centering, using the computational processing system, digit arterial pressure waveform data by subtracting an arterial pressure of the digit arterial pressure waveform data;
scaling, using the computational processing system, the digit arterial pressure waveform data with the selected scale;
uncentering, using the computational processing system, the scaled digit arterial pressure waveform data by adding the arterial pressure of the digit arterial pressure waveform data; and
adding, using the computational processing system, the selected offset to the uncentered and scaled digit arterial pressure waveform data.
Example 21: The method of example 20, wherein the computational processing system is housed within a hemodynamic monitoring system.
Example 22: The method of example 20 or 21, wherein the arterial pressure is one of: a mean arterial pressure, a systolic pressure, or a diastolic pressure.
Example 23: The method of example 20, 21, or 22, wherein the selecting step further comprises: selecting, using the computational processing system, the scale and the offset parameters via a lookup table comprising learned associations between feature vectors and function parameters of scale and offset.
Example 24: The method of example 23 further comprising: training, using the computational processing system, a computational model to generate the lookup table, wherein the training comprises learning associations between a cohort of patient data and radial intra-arterial pressure measurements captured via catheterization, wherein the patient data comprises hemodynamic data obtained from a pressurized digit cuff and physiological data obtained from a photoplethysmogram.
Example 25: The method of example 20, 21, or 22, wherein the selecting step further comprises: selecting, using the computational processing system, the scale and the offset using a regression-based model or a classification-based model.
Example 26: The method of example 25 further comprising: training, using the computational processing system, the regression-based model or the classification-based model by learning associations between cohort of patient data and radial intra-arterial pressure measurements captured via catheterization, wherein the patient data comprises hemodynamic data obtained from a pressurized digit cuff and physiological data obtained from a photoplethysmogram.
Example 27: The method of any one of examples 20 to 26 further comprising displaying, using the computational processing system, the radial arterial pressure waveform data on a display screen.
Example 28: A hemodynamic monitoring system for monitoring radial arterial pressure via captured digit arterial pressure, the system comprising:
a pressurized digit cuff;
a photoplethysmogram; and
a computational processing system in digital connection with the digit cuff and the photoplethysmogram; the computational processing system comprising:
a processor system; and
a memory system comprising one or more applications that can direct the processor system to:
   receive hemodynamic data derived from the pressurized digit cuff and physiological data derived from the photoplethysmogram, wherein the hemodynamic data comprise digit arterial pressure waveform data and the physiological data comprise physiological information of the digit;
   map the hemodynamic data and physiological data to a vector representative of the hemodynamic data and physiological data;
   select a set of transformation function parameters based on the representative vector; and
   transform the digit arterial pressure waveform data into radial arterial pressure waveform data utilizing the selected set of transformation function parameters.
Example 29: The hemodynamic monitoring system of example 28, wherein the one or more applications can direct the processor system to determine the digit arterial pressure data via a volume clamp method.
Example 30: The hemodynamic monitoring system of example 28 or 29, wherein the hemodynamic data and physiological data further comprise one or more of: cardiac output, stroke volume, stroke volume variation, vascular tone, contractility, afterload, systemic vascular resistance, systolic pressure of a digit, diastolic pressure of a digit, mean arterial pressure of a digit, left ventricular ejection time, reconstructed brachial arterial waveform data, pulse pressure, DC, AC,

$$\frac{HR}{fSys} , \frac{fDia}{fMAP} , \frac{fSys - fDia}{fMAP} , \frac{fMAP - fDia}{fSys - fDia} , \frac{dP}{dt} , \frac{AC}{DC} ,$$

or instrumentation data comprising one or more: cuff type, cuff size, or sensor position in relation to heart.
Example 31: The hemodynamic monitoring system of example 28, 29, or 30, wherein the one or more applications can further direct the processor system to determine that a particular data point of the hemodynamic data and physiological data is beyond a limit threshold; wherein the mapping step utilizes a limit value instead of the particular data point.
Example 32: The hemodynamic monitoring system of any one of examples 29 to 31, wherein the mapping step comprises one of the following techniques: principal component analysis (PCA), kernel-PCA, graph-based kernel PCA, the Nyström method, linear discriminant analysis (LDA), generalized discriminant analysis (GDA), T-distributed stochastic neighbor embedding (t-SNE), or uniform manifold approximation and projection (UMAP).
Example 33: The hemodynamic monitoring system of any one of examples 29 to 32, wherein the memory system further comprises a lookup table comprising learned associations between feature vectors and transformation function parameters, and wherein the one or more applications can further direct the processor system to select the set of transformation function parameters via the lookup table.
Example 34: The hemodynamic monitoring system of example 33, wherein the lookup table had been generated via a trained computational model that has been trained on a cohort of patient data comprising hemodynamic data obtained from a pressurized digit cuff, physiological data obtained from a photoplethysmogram, and radial intra-arterial pressure measurements captured via catheterization.
Example 35: The hemodynamic monitoring system of any one of examples 29 to 32, wherein the memory system further comprises a regression-based model or a classification-based model; and wherein the one or more applications can further direct the processor system to select the set of transformation function parameters using the regression-based model or the classification-based model.
Example 36: The hemodynamic monitoring system of example 35, wherein the regression-based model or the classification-based model has been trained on a cohort of patient data comprising hemodynamic data obtained from a pressurized digit cuff, physiological data obtained from a photoplethysmogram, and radial intra-arterial pressure measurements captured via catheterization.
Example 37: The hemodynamic monitoring system of any one of examples 29 to 36, wherein the one or more applications can further direct the processor system to:
determine that a particular parameter of the selected set of transformation function parameters is beyond a limit threshold; and
select an alternative parameter value to replace the particular parameter that is beyond the limit threshold.
Example 38: The hemodynamic monitoring system of example 37, wherein the alternative parameter value is one of: an upper limit value or a lower limit value.
Example 39: The hemodynamic monitoring system of any one of examples 29 to 38, wherein the one or more applications can further direct the processor system to determine that the representative vector is abnormal; and wherein the selected set of transformation function parameters is one of: a previously selected set of transformation function parameters, a set of transformation function parameters having a set of prespecified values, or a combination of previously selected sets of transformation function parameters.
Example 40: The hemodynamic monitoring system of any one of examples 29 to 31, wherein the transformation is performed by a linear transformation and the selected transformation function parameters are a scale and an offset, wherein the one or more applications can further direct the processor system to:
center the digit arterial pressure waveform data by subtracting an arterial pressure of the digit arterial pressure waveform data;
scale the digit arterial pressure waveform data with the selected scale;
uncenter the scaled digit arterial pressure waveform data by adding the arterial pressure of the digit arterial pressure waveform data; and
add the selected offset to the uncentered and scaled digit arterial pressure waveform data.
Example 41: The hemodynamic monitoring system of example 40, wherein the arterial pressure is one of: a mean arterial pressure, a systolic arterial pressure, or a diastolic arterial pressure.
Example 42: The hemodynamic monitoring system of example 41 further comprising a display screen in connection with the processor system, wherein the one or more applications can further direct the processor system to display the radial arterial pressure waveform data on a display screen.
Example 43: A method for training a model to predict a set of transformation function parameters for transforming digit arterial pressure data into radial arterial pressure data, comprising:
receiving, using a computational processing system, noninvasive hemodynamic data and physiological data and radial intra-arterial pressure waveform data from a cohort of patients;
clustering, using the computational processing system, similar data of the noninvasive hemodynamic data and physiological data to yield a plurality of clusters;
averaging, using the computational processing system, the similar data of each cluster;
mapping, using the computational processing system, the averaged data of each cluster to a feature vector representative of the noninvasive hemodynamic data and physiological data of each cluster;
associating, using the computational processing system, the representative feature vectors with the received radial intra-arterial pressure waveform data; and
training, using the computational processing system, a computational model with the representative feature vectors and its associated radial intra-arterial pressure such that the computational model is trained to predict a set of transformation function parameters for transforming digit arterial pressure data into radial arterial pressure data.
Example 44: The method of example 43, wherein the noninvasive digit hemodynamic data is derived from a finger cuff via a volume clamp method.
Example 45: The method of example 43 or 44, wherein the noninvasive digit physiological data is derived from a photoplethysmogram.
Example 46: The method of example 43, 44, or 45, wherein the clustering step further comprises: clustering, using the computational processing system, the similar hemodynamic data by connectivity-based clustering, centroid-based clustering, distribution-based clustering, density-based clustering, grid-based clustering, k-means clustering, or the Louvain method for community detection.
Example 47: The method of any one of examples 43 to 46, wherein the model is a regression-based model or a classification-based model.
Example 48: The method of example 47, wherein the regression-based model comprises LASSO regression, ridge regression, K-nearest neighbors, clastic net, least angle regression (LAR), or random forest regression.
Example 49: The method of example 47, wherein the classification-based model comprises support vector machine (SVM), decision trees, random forests, or naïve Bayes.
Example 50: The method of any one of examples 43 to 49, further comprising generating, using the computational processing system, a lookup table of associations between features vectors and predicted sets of transformation function parameters.

What is claimed is:

1. A real-time method for a hemodynamic monitoring system to transform digit arterial pressure data into radial arterial pressure data, comprising:
obtaining hemodynamic data of a digit via a pressurized digit cuff of a hemodynamic monitoring system, wherein the hemodynamic data comprise digit arterial pressure waveform data;
obtaining physiological data via a photoplethysmogram of the hemodynamic monitoring system, wherein the physiological data comprise physiological information of the digit;
receiving, using a computational processing system of the hemodynamic monitoring system, the hemodynamic data and the physiological data;
mapping, using the computational processing system, the hemodynamic data and physiological data to a representative vector that represents the hemodynamic data and physiological data;
selecting, using the computational processing system, a set of transformation function parameters based on the representative vector, wherein the set of transformation parameters can be utilized to transform the digit arterial pressure waveform data into radial arterial pressure waveform data; and
transforming, using the computational processing system, the digit arterial pressure waveform data into radial arterial pressure waveform data utilizing the selected set of transformation function parameters.

2. The method of claim 1, wherein the hemodynamic data is determined via a volume clamp method using the pressurized digit cuff.

3. The method of claim 1 or 2 further comprising:
determining, using the computational processing system, that a particular data point of the hemodynamic data or a particular data point of the physiological data is beyond a limit threshold; wherein the step of mapping the hemodynamic data and physiological data to a representative vector further comprises: utilizing a limit value instead of the particular data point.

4. The method of claim 1, 2 or 3, wherein the step of selecting a set of transformation parameters further comprises:
selecting, using the computational processing system, the set of transformation function parameters via a lookup table comprising learned associations between feature vectors and transformation function parameters; or
selecting step further comprises: selecting, using the computational processing system, the set of transformation function parameters using a regression-based model or a classification-based model.

5. The method of any one of claims 1 to 4 further comprising:
determining, using the computational processing system, that a particular parameter of the selected set of transformation function parameters is beyond a limit threshold; and
selecting, using the computational processing system, an alternative parameter value to replace the particular parameter that is beyond the limit threshold, wherein the alternative parameter value is one of: an upper limit value or a lower limit value.

6. The method of any one of claims 1 to 5 further comprising:
determining, using the computational processing system, whether the representative vector is abnormal; wherein when the representative vector is abnormal, the step of selecting a set of transformation parameters further comprises one of:
selecting, using the computational processing system, a previously selected set of transformation function parameters;
selecting, using the computational processing system, a set of transformation function parameters having a set of prespecified values; or
selecting, using the computational processing system, a combination of previously selected sets of transformation function parameters.

7. The method of any one of claims 1 to 6, wherein the transforming step is performed by a linear transformation and the set of transformation function parameters comprises a scale and an offset, wherein the method further comprises:
centering, using the computational processing system, the digit arterial pressure waveform data by subtracting an arterial pressure of the digit arterial pressure waveform data;
scaling, using the computational processing system, the digit arterial pressure waveform data with the selected scale;
uncentering, using the computational processing system, the scaled digit arterial pressure waveform data by adding the arterial pressure of the digit arterial pressure waveform data; and
adding, using the computational processing system, the selected offset to the uncentered and scaled digit arterial pressure waveform data to yield the radial arterial pressure waveform data.

8. The method of claim 7 wherein the step of selecting a set of transformation parameters further comprises:
selecting, using the computational processing system, the scale and the offset parameters via a lookup table comprising learned associations between feature vectors and function parameters of scale and offset; or
selecting, using the computational processing system, the scale and the offset using a regression-based model or a classification-based model.

9. The method of any one of claims 1 to 8 further comprising:
displaying, using the computational processing system, the radial arterial pressure waveform data on a display screen of a hemodynamic monitor.

10. The method of any one of claims 1 to 9 further comprising:
determining, using the computational processing system, proximal arterial pressure waveform data from the radial arterial pressure waveform data; and
displaying, using the computational processing system, the proximal arterial pressure waveform data on a display screen.

11. A hemodynamic monitoring system for monitoring radial arterial pressure via captured digit arterial pressure, the system comprising:
a pressurized digit cuff;
a photoplethysmogram; and
a computational processing system in digital connection with the digit cuff and the photoplethysmogram; the computational processing system comprising:
a processor system; and
a memory system comprising one or more applications that can direct the processor system to:
receive hemodynamic data derived from the pressurized digit cuff and physiological data derived from the photoplethysmogram, wherein the hemodynamic data comprise digit arterial pressure waveform data and the physiological data comprise physiological information of the digit;
map the hemodynamic data and physiological data to a representative vector that represents the hemodynamic data and physiological data;
select a set of transformation function parameters based on the representative vector; and
transform the digit arterial pressure waveform data into radial arterial pressure waveform data utilizing the selected set of transformation function parameters.

12. The hemodynamic monitoring system of claim 11, wherein the one or more applications can direct the processor system to determine the digit arterial pressure data via a volume clamp method.

13. The hemodynamic monitoring system of claim 11 or 12, wherein the one or more applications can further direct the processor system to:
determine that a particular data point of the hemodynamic data and physiological data is beyond a limit threshold; wherein the step to map the hemodynamic data and physiological data to a representative vector utilizes a limit value instead of the particular data point.

14. The hemodynamic monitoring system of claim 11, 12, or 13, wherein the memory system further comprises one of:
a lookup table comprising learned associations between feature vectors and transformation function parameters, wherein the step to select the set of transformation function parameters comprises: select the set of transformation function parameters via the lookup table; or
a regression-based model or a classification-based model; the step to select the set of transformation function parameters comprises: select the set of transformation function parameters using the regression-based model or the classification-based model.

15. The hemodynamic monitoring system of any one of claims 11 to 14, wherein the one or more applications can further direct the processor system to:
determine that a particular parameter of the selected set of transformation function parameters is beyond a limit threshold; and
select an alternative parameter value to replace the particular parameter that is beyond the limit threshold, wherein the alternative parameter value is one of: an upper limit value or a lower limit value.

16. The hemodynamic monitoring system of any one of claims 11 to 15, wherein the one or more applications can further direct the processor system to:
determine whether the representative vector is abnormal; wherein when the representative vector is abnormal, the step to select the set of transformation function parameters comprises one of:
select a previously selected set of transformation function parameters;
select a set of transformation function parameters having a set of prespecified values; or
select a combination of previously selected sets of transformation function parameters.

17. The hemodynamic monitoring system of any one of claims 11 to 16, wherein the transformation is performed by a linear transformation and the set of transformation function parameters comprises a scale and an offset, wherein the one or more applications can further direct the processor system to:
center the digit arterial pressure waveform data by subtracting an arterial pressure of the digit arterial pressure waveform data;
scale the digit arterial pressure waveform data with the selected scale;
uncenter the scaled digit arterial pressure waveform data by adding the arterial pressure of the digit arterial pressure waveform data; and
add the selected offset to the uncentered and scaled digit arterial pressure waveform data.

18. The hemodynamic monitoring system of claim 17, wherein the step to select the set of transformation function parameters further comprises one of:
select the scale and the offset parameters via a lookup table comprising learned associations between feature vectors and function parameters of scale and offset; or
select the scale and the offset using a regression-based model or a classification-based model.

19. The hemodynamic monitoring system of any one of claims 11 to 18 further comprising:
a display screen in connection with the computational processing system, wherein the one or more applications can further direct the processor system to:
display the radial arterial pressure waveform data on the display screen.

20. The hemodynamic monitoring system of any one of claims 11 to 19 further comprising:
a display screen in connection with the computational processing system, wherein the one or more applications can further direct the processor system to:
determine proximal arterial pressure waveform data from the radial arterial pressure waveform data; and
display the proximal arterial pressure waveform data on the display screen.
